(19) United States
(12) Patent Application Publication
(10) Pub. No.: US 2017/0169714 A1
(43) Pub. Date: Jun. 15, 2017

(54) Methods and Systems for Cognitive Training Using High Frequency Heart Rate Variability

(71) Applicant: University of Rochester, Rochester, NY (US)

(72) Inventors: Feng Lin, Rochester, NY (US); Mark Mapstone, Irvine, CA (US); Kathi L. Heffner, Rochester, NY (US); Duje Tadin, Pittsford, NY (US); Jiebo Luo, Rochester, NY (US)

(21) Appl. No.: 15/376,223

(22) Filed: Dec. 12, 2016

Related U.S. Application Data
(60) Provisional application No. 62/266,116, filed on Dec. 11, 2015.

(51) Int. Cl. G09B 5/02; A61B 5/00; A61B 5/024; G09B 19/00; A61B 5/16

(52) U.S. Cl. CPC G09B 5/02; G09B 19/00; A61B 5/16; A61B 5/02405; A61B 5/4836; A61B 5/02427; A61B 5/0022

(57) ABSTRACT

Disclosed are systems and methods for administering cognitive training to a subject in need thereof.

This work was supported by grant No. KL2 TR000095 from National Center for Advancing Translational Sciences of the National Institutes of Health. The government has certain rights in the invention.

### I. BACKGROUND

1. Mild Cognitive Impairment (MCI), especially the amnestic type, is considered a symptomatic pre-Alzheimer's disease (AD) phase. Older adults with MCI are a key population to target for interventions aimed at preventing or slowing cognitive decline. Vision-based speed of processing (VSOP) cognitive training is one of the most widely applied behavioral interventions, addressing the cognitive domains of processing speed and attention in community-dwelling older Americans free of AD. Processing speed and attention are key for efficient processing of sensory and cognitive inputs, provide a foundation for multiple cognitive processes (e.g., cognitive control, working memory), cognitive structures (e.g., memory), and everyday functioning, and are among the first domains to show age-dependent declines and predictive of both MCI incidence and progression to AD. What are needed are cognitive training systems and cognitive training regimens that maximize the efficiency and efficacy of VSOP training.

### II. SUMMARY

2. Disclosed are cognitive training systems for administering cognitive training comprising a computer, a training module specifically designed to administer a cognitive training program and receive training data; a display for administration of the training program, an input device for receiving patient training data, a portable high frequency variable heart rate monitor, a receiver configured to receive input from the variable heart rate monitor, a communication module specifically designed to convert the signal from the monitor into useable input data for use in the training program; wherein the cognitive training system continually adjusts the training based on input from the high frequency variable heart rate monitor to maximize cognitive plasticity.

3. Also disclosed are methods of treating a subject with mild cognitive impairment comprising administering cognitive training to the subject; measuring high frequency variable heart rate of the subject, correlating the high frequency variable heart rate measurement with the neural plasticity of the subject, modulating the difficulty of the cognitive training to optimize plasticity of the subject induced by the cognitive training.

### III. BRIEF DESCRIPTION OF THE DRAWINGS

4. The accompanying drawings, which are incorporated in and constitute a part of this specification, illustrate several embodiments and together with the description illustrate the disclosed compositions and methods.

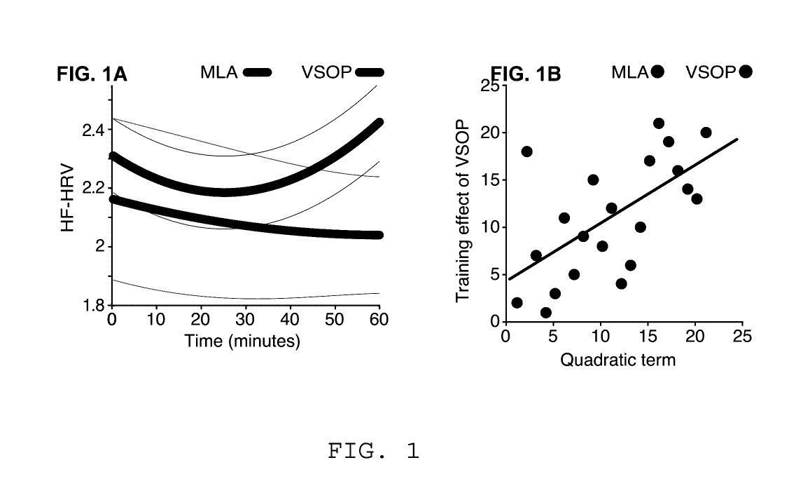

5. FIG. 1 shows the effects of VSOP training on HF-HRV. FIG. 1 A shows Modeling HF-HRV over training session using the quadratic model: Y_{HF-HRV}=aXₜᵢₘₑ²+bXₜᵢₘₑ +c. Bold lines show the group average. Thin lines are SEM. FIG. 1B shows a comparison of model terms (a, b & c). The key group difference was in the quadratic term. ** p<0.01; §p<0.06).

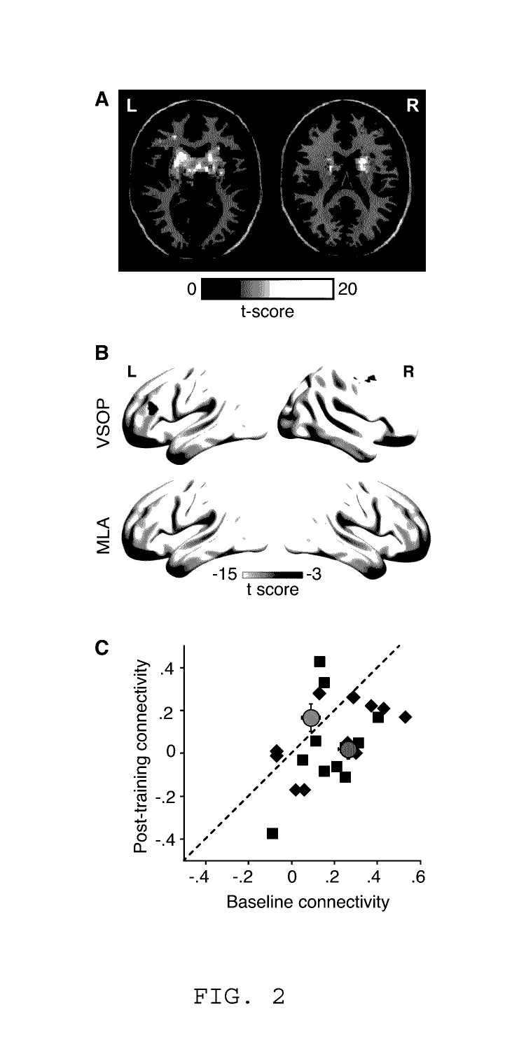

6. FIG. 2 shows the effects of VSOP training on bilateral striatum-prefrontal networks. FIG. 2A shows bilateral striatum-prefrontal networks at baseline: L=left, R=right. FIG. 2B shows training-induced changes in striatum-prefrontal networks for the VSOP group: L, left striatum-left inferior prefrontal gyrus (−45, 27, 21); R, right striatum-right middle frontal gyrus (42, 6, 60). No changes were found for the MLA group. FIG. 2C shows changes in bilateral stratum-prefrontal connectivity from baseline to post-training. Squares show left striatum-prefrontal network. Triangles show right striatum-prefrontal network. Circles show group averages. Error bars are SEM. Changes in the striatum-prefrontal networks were correlated with the quadratic term of HF-HRV responses (Left: r=0.41, 95%CI: 0.19, 0.91; Right: r=0.55, 95%CI: 0.39, 0.93).

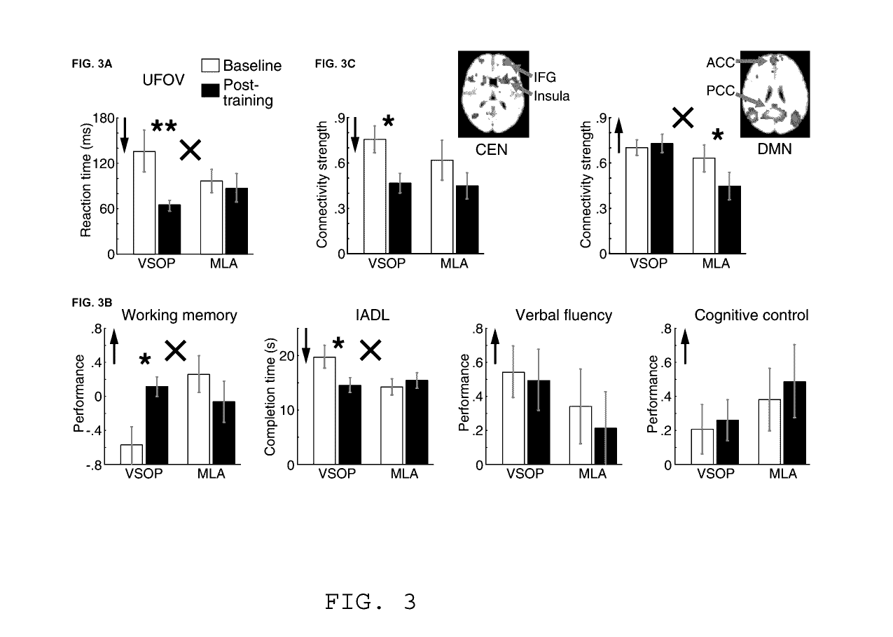

7. FIG. 3 shows the effects of vision-based speed-of-processing (VSOP) training and mental leisure activities (MLA) control training on a range of cognitive and neural domains. (A) Effects of training on useful field of view (UFOV), the trained domain for VSOP training. (B) Effects of training on transfer domains: working memory, instrumental activities of daily living, verbal fluency, and cognitive control. (C) Effects of training on neural domains: resting state neural connectivity for the central executive network (CEN) and default mode network (DMN); inserts show horizontal brain slices that include regions of interest for each network (IFG=inferior frontal gyrus, ACC=anterior cingulate cortex, PCC=posterior cingulate cortex). Higher scores indicated better outcome; Lower scores indicated better outcome. Within-group (baseline vs after training) comparison: P<*0.05, **0.01. Group (VSOP vs MLA) by time (baseline vs after training) comparison: 9P<0.05.

### IV. DETAILED DESCRIPTION

8. Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

9. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

10. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

11. In this specification and in the claims which follow, reference will be made to a number of terms which shall be defined to have the following meanings:

12. “Optional” or “optionally” means that the subsequently described event or circumstance may or may not occur, and that the description includes instances where said event or circumstance occurs and instances where it does not.

13. Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

14. Many research studies have shown that targeted training improves the trained cognitive or functional abilities, but the effects of training on untrained abilities (i.e., transfer effects) have not been reliably demonstrated. Herein, is shown that mental fatigue in older adults can affect the amount of benefit from cognitive training in addition to the level of transfer to other non-trained tasks and functional abilities. Additionally, it is shown that parasympathetic control of the autonomic nervous system, which can be measured using high frequency heart rate variability (HF-HRV), respiration, baroreflex assessment, neurotransmitters, thermoregulation, cardiovascular reflexes, valsalva maneuver, apneic facial immersion, spectral analysis, and/or cardiovascular response, and in particular, patterns of changes in heart rate during lying down or squatting, can provide an accurate and sensitive surrogate of the reactivity to cognitive load during cognitive training tasks. For example, certain non-linear patterns of HF-HRV, which reflects an adaptive flexible response to the environment, over a cognitive training session can predict the plasticity/gain of the brain from the training tasks, as well as the transferred training effects. Therefore, a measured parasympathetic pattern (such as, for example, HF-HRV pattern) over a cognitive training session can reflect how much an older adult can benefit from the cognitive training, which can be altered during the session to optimize the training effect.

15. In one aspect, the disclosure divulged herein provides a new methodology that manipulates the difficulty of cognitive training tasks in real time from parasympathetic measures (e.g., HF-HRV, respiration, baroreflex assessment, neurotransmitter s, thermoregulation, cardiovascular reflexes, valsalva maneuver, apneic facial immersion, spectral analysis, and/or cardiovascular response, and in particular, patterns of changes in heart rate during lying down or squatting) recorded simultaneously by a parasympathetic measuring device (including, for example, a HF-HRV monitor), such as for example, electrocardiography (ECG) and systems for implementing the same. The development of this methodology consists of two steps: development of the cognitive training task paradigms that are most compatible (i.e. sensitive and beneficial) with HF-HRV activities and analysis of various HF-HRV patterns, as well as their associations with cognitive training task performance to determine the indices that help maximize plasticity and the transferred training effect. This cognitive training task is a key component of a portable human-machine interface device, which simultaneously assesses heart activities and transmit this information wirelessly (e.g. Bluetooth, cellular data, infrared signal, radio wave, ultrahigh frequency transmission (UHF), and/or very high frequency transmission (VHF)), in real time, to the cognitive training software running on a computer (desktop, portable, or smartphone), while the subject is engaged in the training paradigm for direct modulation of the training paradigm.

16. In one aspect, disclosed herein are training systems for administering cognitive training (i.e., cognitive training systems). Through monitoring parasympathetic nervous system (including, HF-HRV, respiration, baroreflex assessment, neurotransmitter s, thermoregulation, cardiovascular reflexes, valsalva maneuver, apneic facial immersion, spectral analysis, and/or cardiovascular response, and in particular, patterns of changes in heart rate during lying down or squatting), the skilled practitioner can modulate the conditions of the cognitive training to maximize the benefit of the training. Thus, in one aspect, disclosed herein are cognitive training systems comprising a high frequency heart rate variability monitor (HF-HRV).

17. It is understood and herein contemplated that there are many means through which a monitor may measure high frequency heart rate variability. In one aspect the monitor can comprise invasive (e.g., catheter-based), low-fidelity electrical measures (for example, a 12-lead ECG) or noninvasive measures. There are many equally sufficient methods for noninvasively monitoring, measuring, or obtaining the HF-HRV of a subject which may be used alone or in conjunction including, but not limited to, electrocardiography (ECG), electrocardiographic imaging (ECGI), magnetic resonance imaging (MRI), nuclear medicine studies (PET, SPECT), computed tomography (CT) scanning, cardiac ultrasonography (i.e., echocardiography), photoplethysmography, acoustic imaging, colorimetric imaging, pressure imaging, or any other noninvasive imaging technique known. Accordingly, in one aspect, disclosed herein are cognitive training systems comprising a noninvasive means for monitoring, measuring, or obtaining the HF-HRV of a subject, wherein the noninvasive imaging means comprises one or more of electrocadiagraphic imaging, magnetic resonance imaging, cardiac computed tomography, cardiac nuclear medicine, cardiac ultrasonoagraphy, colorimetric imaging, acoustic imaging, pressure imaging, and photoplethysmography.

18. “Electrocardiography” (ECG) refers to process of recording the electrical activity of the heart (such as, for example, cardiac electrical potential) over a period of time using electrodes placed on a subject's body. The measurement can utilize 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, or 300 or more electrodes to measure electrical changes associated with each heartbeat. Thus, in one aspect, disclosed herein are cognitive training systems wherein the variable heart rate monitor comprises one or more electrode leads for measuring cardiac electrical potential.

19. Electrocardiographic Imaging (ECGI) is an ECG variant and is an important development toward improving four-dimensional precision of imaging cardiac electrophysiology. As used herein, “electrocardiographic imaging” (ECGI) refers to a technique which reconstructs epicardial potentials, electrograms, and activation sequences (isochrones) from electrocardiographic body-surface potentials noninvasively. It is similar to CT or MRI, except that it is designed to image cardiac electrical function. The technique addresses solving of the electocardiographic inverse problem, which due to computation of epicardial potentials from body surface potentials cab result in significant errors, through the use of Tikhonov regularization or the generalized minimal residual (GMRes) method. Typically ECGI utilizes (i) electrocardiographic unipolar potentials measured over the entire body surface (BSPs) and (ii) the heart-torso geometrical relationship.

20. In one aspect, ECGI can utilize an electrode vest strapped to the subject's torso and connected to a multichannel system measured BSP. The vest can include at least 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, 180,190, 200, 210, 220, 230, 240, 250, 260, 270, 280, 290, or 300 or more electrodes to measure BSP. ECGI incorporates the patient-specific anatomy of the heart with the recording leads on the body surface to noninvasively reconstruct the electrical activity (including, but not limited to high frequency variable heart rate) on a three-dimensional model of the patient's heart surface.

21. As used herein, “magnetic resonance imaging” refers to refers to the use of use magnetic fields and radio waves to form images of the body. Typically, when used in cardiac situations, cardiovascular magnetic resonance imaging (CMR) involves ECG gating which combats the artifacts created by the beating of the heart.

22. “Cardiac computed tomography” means the use of x-ray images taken from the patient at different angles to produce tomographic (cross-sectional) images.

23. Another means for imaging an HF-HRV includes echocardiography also known as “cardiac ultrasonoagraphy.” As used herein, “cardiac ultrasonoagraphy” means the use of uses standard two-dimensional, three-dimensional, and/or Doppler ultrasound to create images of the heart.

24. As used herein, “photoplethysmography” refers to an optically obtained volumetric measurement of an organ. As blood is pumped through a body the diameter of the arteries, veins, and capillaries change with the influx and outflux of blood through the vessel, a process referred to as vasodilation. A photoplethysmographic device typically comprises a light source (i.e., an emitter, such as, a light emitting diode (LED)) and an optical sensor such as, for example, a photodetector. The photoplethysmographic device emits light from the emitter and measures the amount of light reflection and/or absorption that is received by the photodetector. The emitter can comprise one, two, three, four, five, six, seven, or more light emitters with each emitting a different wavelength of light. Typically, the emitter emits two different wavelengths of light and determines vasodilation based on the different rate of absorbance and/or reflectance for each wavelength. The photoplethysmographic device can be affixed to a clamp and secured on an earlobe or finger or affixed to a body part by adhesive or a strap and affixed to any limb, the head, or chest of a subject. Thus, in one aspect, disclosed herein are cognitive training systems wherein the variable heart rate monitor comprises a light source and an optical sensor to measure light absorbance or reflectivity of the light off of capillaries in the subject (i.e., a photoplethysmographic device). Examples of photoplethysmographic devices include but are not limited to pulse monitors, pulse oximeters, and biomonitors.

25. As used herein “pressure imaging” refers to the use of the pressure associated with vasodilation. In one aspect, the pressure imaging can be obtained by use of a piezoelectric sensor, piezoresistive strain gauge, capacitive pressure sensor, and/or optical pressure sensors using Fiber Bragg Gratings. Pressure imaging can detect HF-HRV by measuring the vibration associated with vasodilation. In the case of a piezoelectric sensor, the vibrations at the sensor generate and electrical signal which can be interpreted in the same way an ECG is interpreted to display an HF-HRV.

26. As used herein, “acoustic imaging” refers to the use of a microphone to transmit an acoustic based electrical signal from the sound of vasodilation. In one aspect, the sound of vasodilation causes a transducer in a microphone to vibrate which generates an electrical signal which can then be converted back to an audio signal through a second transducer or interpreted via a processor to a representation of a heart

27. As used herein “colorimetric imaging” refers to the use of a light source and an optical sensor to measure changes in color in the skin as blood flows through the capillaries with each heartbeat. In one aspect, the colorimetric imaging can be obtained where the light source and optical sensor are in the same device or separate. In one aspect, the colorimetric imaging device can be a mobile device such as a smart phone or camera.

28. In one aspect, it is understood that the use of ECG, ECGI, pressuring imaging, acoustic imaging, colorimetric imaging, or photoplethysmographic devices can be accomplished in a portable format such that the HF-HRV data can be compiled while the subject is at rest or moving but not physically connected to any device other than the HF-HRV monitor and optionally a separate transmitter. For example, the HF-HRV monitor can be a wearable device such as, for example, a watch, chest band, head band, vest, shirt, jacket, wrist band, or arm band. In one aspect, the HF-HRV monitor can be a portable electronic device with video, photo, or other imaging capabilities such as a smart phone, tablet computer, laptop computer, or camera.

29. The high frequency hear rate variability monitor obtains HF-HRV data, but then must transmit this data to the training module so the cognitive training can be adjusted to optimize plasticity of the subject undergoing cognitive training. It is contemplated herein that measurements taken by the monitor can be transmitted to a receiver that supplies the monitor information to a cognitive training module. The transmission of HF-HRV may be done via direct connection (i.e., wired) or via wireless transmission. As used herein, wireless transmission can be accomplished through any appropriate manner in which data may be transmitted including, but not limited to ultrasonic transmission, infrared transmission, free space optical transmission, Bluetooth transmission, ANT transmission, cellular transmission (including, but not limited to, Global System for Mobile Communications (GSM), Code Division Multiple Access (CDMA), Universal Mobile Telecommunications System (UMTS), Enhanced Data rates for GSM Evolution (EDGE), General packet radio service (GPRS), High Speed Packet Access (HSPA)), electromagnetic transmission, and radio transmission (including, but not limited to High Frequency (HF) band, Very High Frequency (VHF) band, Ultra High Frequency (UHF) band, industrial, scientific and medical (ISM) radio bands, and Super High Frequency (SHF) Band).

30. In one aspect tramission of the HF-HRV data can be accomplished through the use of a transmitter configured to transmit the monitor data to a receiver which is connected or integral to a cognitive training module. Thus, in one aspect, disclosed herein are cognitive training systems, wherein the HF-HRV monitor further comprises a transmitter module which receives the input from the monitor and transmits them to the receiver. It is understood and herein contemplated that the transmitter can be a physically separate component of the cognitive training system or integral to the HF-HRV monitor. Where a separate component than the HF-HRV, the monitor and the transmitter can be connected via leads.

31. As disclosed above, the transmission of the HF-HRV data can be received by a receiver configured to receive HF-HRV data input from the HF-HRV monitor. The receiver can receive wireless transmission of HF-HRV data or direct data input. Thus, in one aspect, disclosed herein are cognitive training systems wherein the receiver has inputs to receive signals from the HF-HRV monitor. It is understood and herein contemplated that the receiver can be a separate physical component of the cognitive training module or integral to said module.

32. Once the receiver has received the HF-HRV data, it is contemplated herein that a communication module can be utilized to convert the signal from the monitor into a usable input data form for use with the cognitive training program. In one aspect, the communication module can be a separate component of the cognitive training system or a component of the receiver and/or the training module. In one aspect, the communication module processor specifically designed to HF-HRV data from the receiver into usable input data. The input data can be displayed on a graphical monitor, printed, and/or combined with indices of cognitive performance (reaction time and/or accuracy rate).

33. In one aspect, it is contemplated herein that the cognitive training systems and methods disclosed herein can comprise a processor specifically designed to implement a HF-HRV adaptive algorithm that combines the HF-HRV data that has been converted to a usable input data with cognitive performance data and outputs an HF-HRV pattern that reveals the efficacy of the cognitive training. The processor for implementing the HRV adaptive algorithm can be the same or different processor than the processor that converts the HF-HRV data into usable input data. In one aspect, the algorithm can employ multiple indices for heart rate variability including, but not limited to low frequency (LF)-heart rate variability (HRV), HR-HRV, and/or LF/HF HRV ratio, as well as indicators of the difficulty of the cognitive training based on accuracy rate and/or reaction time. From these indices, a HF-HRV patter can be generated. The algorithm can use a machine learning approach, e.g., prediction based on multivariate multiple regression, in response to the mined HF-HRV patterns. For example, the HF-HRV pattern can be a U-shaped curve when HF-HRV is plotted against time. Where a flat curve indicates cognitive training that is too simple and a U-shape that forms in less than 1, 2, 3,4, 5, 6, 7, 8, 9, 10, 11, 12, 13, 14, 15, 16, 17, 18, 19, 20, 21, 22, 23, 24, or 25 minutes indicates that the cognitive training is too difficult. The Computerized Cognitive Training (CCT) program can determine the next suitable difficulty level or type of task on the fly in a manner that maintains HF-HRV in or changes it to desired patterns over 70% of time.

34. It is understood and herein contemplated that the cognitive training system can further comprise a processor to adjust the difficulty or task rate to maximize cognitive training as reflected the HF-HRV pattern (adjustment processor). Such a processor can provide fully automated adjustments, or output information allowing for manual manipulation of the cognitive training by a qualified practitioner. For example, where the HF-HRV pattern is flat, the processor can adjust the rate of training to be shorter and/or training to be more difficult. Conversely, for example, where the HF-HRV pattern produces a U-shape curve in 1, 2, 3,4, 5, 6, 7, 8, 9, 10, 11, 12, 13, 14, 15, 16, 17, 18, 19, 20, 21, 22, 23, 24, or 25 min or less, the difficulty of training can be decreased or the rate of training increased (i.e., made slower). In one aspect the adjustment processor and processor for applying the algorithm are the same processor. In another aspect, the adjustment processor and processor for applying the algorithm are different processors.

35. In one aspect, the cognitive training system does not employ an adjustment processor. Rather a qualified practitioner (i.e., a practitioner such as a physical therapist, occupational therapist, nurse, physician, physician assistant) can view the HF-HRV pattern or converted HF-HRV data and cognitive performance data (reaction time and/or accuracy rate) on a printout or visual display and manually adjust the cognitive training. For example, where the HF-HRV pattern is flat, the practitioner can adjust the rate of training to be shorter and/or training to be more difficult. Conversely, for example, where the HF-HRV pattern produces a U-shape curve in 1, 2, 3,4, 5, 6, 7, 8, 9, 10, 11, 12, 13, 14, 15, 16, 17, 18, 19, 20, 21, 22, 23, 24, or 25 min or less, the difficulty of training can be decreased or the rate of training increased (i.e., made slower). Thus, in one aspect, the cognitive training system comprises manual controls to adjust the rate and difficulty of the cognitive training and/or a display or printer to reveal the HF-HRV pattern to the qualified practitioner.

36. It is understood and herein contemplated that the disclosed cognitive training system comprises, in one aspect, a cognitive training module specifically configured to administer a cognitive training program. The cognitive training program can be any known computer program or multiple programs comprising instructions related to cognitive training. For example, the cognitive training program may be plasticity-based computer cognitive training (e.g., speed of processing training (including, but not limited to vision based speed of processing and aural speed of processing), working memory training, attention training, perception training (for example, eye tracking), biofeedback training, brain machine interfaces, and phonological awareness programs. Any one or more of the programs can be implemented during the course of training.

37. The disclosed cognitive training can be administered in a group setting or individually. Furthermore, the disclosed cognitive training can be administered at a dedicated device or via the intranet or other web-based system. In one aspect, the cognitive training module of the cognitive training system continually adjusts the training based on input from the high frequency variable heart rate monitor to maximize cognitive plasticity.

38. These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

39. The methods and systems that have been introduced herein, and discussed in further detail, have been and will be described as comprised of units. One skilled in the art will appreciate that this is a functional description and that the respective functions can be performed by software, hardware, or a combination of software and hardware. A unit can be software, hardware, or a combination of software and hardware. In one exemplary aspect, the units can comprise a computer. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

40. The present methods and systems can be operational with numerous other general purpose or special purpose computing system environments or configurations. Examples of well-known computing systems, environments, and/or configurations that can be suitable for use with the cognitive training systems and methods disclosed herein comprise, but are not limited to, personal computers, server computers, laptop devices, cloud services, mobile devices (e.g., smart phones, tablets, and the like) and multiprocessor systems. Additional examples comprise set top boxes, programmable consumer electronics, network PCs, minicomputers, mainframe computers, enterprise servers, distributed computing environments that comprise any of the above systems or devices, and the like. Thus, in one aspect are cognitive training systems comprising a cognitive training module wherein the cognitive training module is resident on a computer.

41. The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, etc., that perform particular tasks or implement particular abstract data types. The disclosed methods can also be practiced in grid-based and distributed computing environments where tasks are performed by remote processing devices that are linked through a communications network. In a distributed computing environment, program modules can be located in both local and remote computer storage media including memory storage devices.

42. The cognitive training system utilize displays so the subject undergoing the training can participate in the training. Such displays may be visual (for example, a computer monitor) or aural (for example, speakers). Accordingly, and in one aspect, disclosed herein are cognitive training systems comprising a display for a computer. It is understood and herein contemplated that the display can be a separate component from the computer of the cognitive training system or integral to a computer. For example, the display can be speakers or a visual monitor that are resident on a computer such as on a laptop, wireless smartphone, or tablet computer. The display may also be speakers and/or a visual monitor that are connected to the computer via direct coupling of wiring that transmit the visual and/or aural information or wirelessly via Bluetooth transmission, ANT transmission, cellular transmission or the like.

43. Cognitive training systems can also comprise input devices for receiving patient training data. Such input devices can include mechanical or optical devices including but not limited to keyboards, cameras, eye trackers, webcams, gamepads, joysticks, trackballs, lightpens, touchscreens, computer mice, microphones, and keyboards, as well as, input devices specifically configured for the recording of responses to the cognitive training.

44. It is understood and herein contemplated that the disclosed cognitive training system can be used for the treatment of subject with cognitive disorders. Accordingly, and in one aspect, disclosed herein are methods of treating a subject with a reading or cognitive disorder comprising administering cognitive training to the subject, measuring high frequency heart rate variability of the subject, correlating the high frequency heart rate variability measurement with the neural plasticity of the subject (for example, creating a HF-HRV pattern), modulating the difficulty of the cognitive training to optimize plasticity of the subject induced by the cognitive training. In some aspect, the method further comprises measuring the cognitive performance (reaction time and/or accuracy rate) and combining the cognitive performance data and HF-HRV measurement data to produce the neural plasticity measurement.

45. Reading and cognitive disorders that can be treated with the disclosed methods and systems can take many forms including, but not limited to reading and cognitive involved in impairments like dyslexia, attention deficit disorder, amblyopia, autism, schizophrenia, dementia, mild-cognitive impairment, Alzheimer's disease and other types of dementia (e.g., vascular dementia, Parkinson's dementia, Lewy body dementia, frontotemporal dementia), stroke, ischemic-infarction, Traumatic Brain Injury (TBI), major depression, multiple sclerosis, and age related loss of cognition. Thus in one aspect, disclosed herein are methods of treating a subject with cognitive disorder comprising administering cognitive training to the subject, wherein the cognitive disorder comprises reading or cognitive disorder involved in impairments like dyslexia, attention deficit disorder, amblyopia, autism, schizophrenia, dementia, mild-cognitive impairment, Alzheimer's disease and other types of dementia (e.g., vascular dementia, Parkinson's dementia, Lewy body dementia, frontotemporal dementia), stroke, ischemic-infarction, Traumatic Brain Injury (TBI), major depression, multiple sclerosis, and age related loss of cognition.

46. As disclosed herein, the treatment of the cognitive disorder can implement utilize any portion or the entirety of any of the cognitive training systems disclosed herein. It is further understood that the form of cognitive training implemented by the cognitive training system for treatment of the cognitive disorder can be any one or more cognitive training method disclosed herein including but not limited to plasticity-based computer cognitive training (e.g., speed of processing training (including, but not limited to vision based speed of processing and aural speed of processing), working memory training, attention training, perception training (for example, eye tracking), biofeedback training, brain machine interfaces, and phonological awareness programs. Thus in one aspect, disclosed herein are methods of treating a subject with cognitive disorder comprising administering cognitive training to the subject, wherein the cognitive training program comprises one or more of plasticity-based computer cognitive training (e.g., speed of processing training (including, but not limited to vision based speed of processing and aural speed of processing), working memory training, attention training, perception training (for example, eye tracking), biofeedback training, brain machine interfaces, and/or phonological awareness programs.

47. In one aspect, the treatment of the cognitive disorder can be adjusted in real time to maximize the plasticity of the subject. Such adjustment can be automated by the cognitive training program or manipulated by a qualified practitioner (i.e., a practitioner such as a physical therapist, occupational therapist, nurse, physician, physician assistant, or other technician trained to understand how to administer the cognitive training test and how to appropriately adjust training based on the HF-HRV measurements) suitable for administering a cognitive training test. Accordingly, in one aspect, disclosed herein are methods of treating a subject with a reading or cognitive disorder comprising administering cognitive training to the subject, measuring high frequency heart rate variability of the subject, correlating the high frequency heart rate variability measurement with the neural plasticity of the subject, modulating the difficulty of the cognitive training to optimize plasticity of the subject induced by the cognitive training, wherein the high frequency heart rate variability measurement and adjustments to the difficulty of the cognitive training are performed continuously throughout the training. For example, HF-HRV of a continuous deepened suppression (i.e., responding to a challenging situation) and followed by a rebound (i.e., enhancing the brain capacity) predicts positive learning and neural plasticity. Where the cognitive training program detects a relatively flat HF-HRV pattern continues for several minutes (indicating the program being too easy), the program would increase its difficult level (by shortening the stimuli appearing time, or changing to a more difficult content). The cognitive training program can constantly detect HF-HRV patterns and adjust the difficult level to ensure participant maintain the effective HF-HRV pattern constantly.

48. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### The Parasympathetic Nervous System in Cognitive Training

49. Amnestic mild cognitive impairment (aMCI) is considered a symptomatic pre-Alzheimer's disease (AD) phase. Thus, older adults with aMCI constitute a key target for interventions aimed at preventing or slowing cognitive decline. Vision-based speed of processing (VSOP) training can result in significant improvements in both trained (i.e., processing speed and attention) and untrained (i.e., working memory and instrumental activities of daily living) cognitive domains. What was previously unknown, however, is what neurophysiological mechanisms account for the impacts of VSOP training in older adults with aMCI. Here, the role of autonomic nervous system in VSOP-induced plasticity was investigated. The work shown herein indicates that VSOP training can also be effective in older adults with MCI. Emerging evidence in healthy younger adults indicates that VSOP training can induce neuroplasticity (i.e., the brain's ability to undergo beneficial restructuring or reprogramming in response to environmental stimuli). Notably, neuroplasticity can be induced in later life, even in MCI. Thus, VSOP training can promote neuroplasticity and slow neurodegeneration in MCI.

50. According to the Neurovisceral Integration Model and recent meta-analyses, a core set of brain regions, most prominently striatum, are involved in links between adaptive cognitive and peripheral physiological regulation. The autonomic nervous system serves a role in this link by connecting the brain and peripheral functions, such as heart rate, in efforts to flexibly adapt to the environment. Of note, such flexible adaptation to cognitive stimuli can be maintained even in the early stages of cognitive decline. In response to environmental stimuli, such as cognitive training, a dynamic neurophysiological regulatory process occurs that promotes ongoing regulation and adaptation to the stimuli.

51. Herein it is shown that parasympathetic activation of autonomic nervous system (PNS) indexed by high frequency heart rate variability (HF-HRV) is a key marker for such regulation and adaptation. This is consistent with a long-standing idea that stimulation of PNS may directly lead to cognitive and memory improvements through the release of cholinergic transmitters. Meanwhile, PNS activation, by providing feedback to striatum that is a hub connecting frontal and posterior cortices as well as subcortical regions, can also lead to large-scale brain changes. It is shown herein that the flexible HRV regulation explains the broad impact of VSOP training.

52. To address this question, VSOP training was compared to an active control (mental leisure activities, MLA) in older adults with aMCI and examined the link between HF-HRV regulation and neuroplasticity caused by VSOP training. Specifically, it is shown herein that, compared to MLA control, VSOP training induces more flexible adaptation of HF-HRV during the training session. Also examined was the role of striatum-related neural network, with a hypothesis that, like HF-HRV, greater changes of striatum is linked to stronger cognitive changes.

a) Methods

(1) Design

53. A randomized controlled trial was conducted. Participants with aMCI were recruited from University of Rochester Memory Care Program using the clinical diagnosis of “mild cognitive impairment due to Alzheimer's disease.” All participants had deficits in memory and executive function based on a comprehensive neuropsychological battery but intact basic activities of daily living, and absence of dementia using NINCDS-ADRDA criteria per assessments. If an individual was on Alzheimer's disease medication (i.e., memantine or cholinesterase inhibitors), it was required to have no changes in dosage in the 3 months prior to recruitment. Participants needed to have capacity to give consent based on clinician assessment and adequate visual acuity for testing, as well as be ≧60 years of age, English-speaking, and community-dwelling. We excluded individuals who had active participation in another cognitive intervention study or active treatment with antidepressants or anxiolytics. 24 participants were enrolled and randomly assigned to the VSOP or MLA group after informed consent and baseline assessment. Cognitive function (processing speed and attention, measured by the Useful Field of View (UFOV); working memory, and instrumental activities of daily living measure by the timed instrumental activities of daily living (TIADL)) and imaging data were assessed at baseline and post-training. Electrocardiography (ECG) was assessed at two in-lab training sessions during the 2^{nd} and 3^{rd} week of the training. Interviewers were blinded to the participants' group assignment. A total of 10 participants from VSOP group and 11 from MLA group completed the study. The study was approved by the University of Rochester Research Subject Review Board.

54. Measures of cognitive outcomes as well as imaging data collection and processing were described elsewhere. In the present study, for the cognitive outcomes with significant group and time interaction effect (UFOV, working memory, and TIADL), the changes were calculated between post-training and baseline. Greater scores in working memory and smaller scores in UFOV indicated positive changes after training.

(2) VSOP Training

55. VSOP training included five computerized attention tasks with visual stimuli (Eye for Detail, Peripheral Challenge, Visual Sweeps, Double Decision, and Target Tracker). In the Eye for Detail task, a series of stimuli (e.g., butterflies) were briefly presented at the same time. Participants needed to identify a number of stimuli that were identical to each other. As the difficulty level increased, the stimuli became more similar to each other. In the Peripheral Challenge task, a number of birds were briefly presented in peripheral vision, including a target bird that was different from other distracter birds. The participants were asked to point out the location of the target bird. As the difficulty level increased, the target and distractor birds became more similar. In the Visual Sweeps task, two sweep patterns were presented simultaneously, and the participants indicated whether the sweeps were moving IN or OUT. In the Double Decision task, a target vehicle was presented in the center of the screen and a road sign was presented in the periphery. Participants needed to determine both the type of vehicle and the location of the road sign. With increases in difficulty level, the vehicles became more similar, and distracters were added. In the Target Tracker task, a number of target jewels were presented on the screen first, and then a number of identical distracter jewels were presented. All of the target and distracter jewels then moved in a Brownian motion fashion for a short period. Upon the pause of the movement, participants needed to pick the target jewels. As task difficulty increased, participants were required to simultaneously track more target jewels and the distracters would become more similar to the targets. Across tasks, participants identified the object and/or location of the object on the screen. To ensure the participants to always operate near their optimal capacity, the training would automatically adjust the task difficulty and speed, and switch the tasks based on the participant's performance.

(3) MLA control

56. MLA control included three computerized vision-based activities (crossword, Sudoku, and solitaire) to control for computer experience and amount of time, and to stimulate participants' everyday mental activities. Training in each group lasted for 6 weeks.

57. Cognitive testing and resting-state imaging data were assessed at baseline and post-training at week 7.

(4) Cognitive Testing

58. Cognitive testing included measurements of the Useful Field of View (UFOV) (trained effect) and working memory (transferred effect). We focused on these two domains because these were the tasks for which we observed significant VSOP training-induced improvements. Examining group (VSOP vs. MLA) by time (baseline vs. 7 week) interaction, VSOP group improved significantly in UFOV (F_{1,19}=6.61, partial η²=0.26, p=0.26) and working memory (F_{1,19}=7.33, partial η²=0.28, p=0. 01) compared to MLA group. Here, we asked if these training-induced improvements could be linked with HF-HRV. UFOV is a measure for processing speed and attention (Visual Awareness Research Group, Inc.), which are the primary domains targeted in the VSOP training, consisting of three subtests to detect, identify, and localize briefly presented targets in the center (subtest 1), in both the center and periphery (subtest 2), and in both the center and periphery with embedded distractors (subtest 3). UFOV is conceptually similar to the Double Decision Task in the VSOP training but uses different tasks and stimuli from the training paradigms. Working memory was assessed using two tasks—dot counting (requires participants to count a series of slides with various numbers of dots and remember the sequence of the number) and 1-back tasks (participants need to determine if the location of the object matches the previously shown location) from the EXAMINER package. Different stimuli were used for the two tasks between baseline and week 7 to avoid practice effect. Changes in UFOV and working memory from baseline to 7 weeks were calculated in the analysis with higher change values indicating more positive changes.

(59.) Development of the Cognitive Training Task Paradigm

59. Development of the cognitive training task paradigm that is most compatible (i.e., sensitive and beneficial) with HF-HRV activities: The linear and nonlinear (e.g., quadratic) modeling of HF-HRV data over the training tasks is computed based on each individual cognitive task paradigm using linear mixed modeling. Indices from these models are compared between and within the task paradigm using ANOVA and proper post hoc analysis. Next, the various HF-HRV patterns, as well as their associations with cognitive training task data are analyzed to confirm the indices that help maximize plasticity and the transferred training effect: ERP (focusing on P3 and N2pc wave attitudes) from EEG can be analyzed to identify the plasticity induced from the cognitive training using paired t test. Indices of HF-HRV from the first step are correlated to the change of ERP using Pearson's r correlation.

(6) Electrocardiography Protocol and Data Reduction

60. Electrocardiography (ECG) was assessed at two in-lab training sessions during the 2^{nd} and 3^{rd} week of the training. The time point was chosen to balance the adequate understanding of the training procedures and novelty of the training content. That is, the protocol was designed to capture HRV after subjects became reasonably familiar with the training tasks, but before task-specific expertise begins to accumulate. Electrocardiography data were collected continuously, using a standard lead-II electrode configuration, at 1000 Hz with a BioNex Mainframe with ECG module (MINDWARE®, LTD). HF-HRV was derived by spectral analysis of the interbeat interval collected from ECG using Mindware software (MINDWARE®, LTD), obtaining total variance within the respiratory range (0.15-0.5 Hz). HF-HRV values were derived over twenty second sampling intervals, and the average HF-HRV was aggregated over the last minute of baseline and across each minute of the 60-minutes of each cognitive training session. Finally, corresponding minutes from the two training sessions were averaged. These aggregate, minute-by-minute HF-HRV values were log-transformed and used in the following analysis.

61. To examine the group difference in HF-HRV during in-lab training sessions, the mixed-effects model was used to model the quadric time structure with equation among individual participants as follows: Y _{HF-HRV}=aX_{Time}²+bX_{Time}+c+group(aX_{Time}²+bX_{Time}+c) +ε where parameters (a, b, c) are modeled as random-effects at individual and group levels. The quadratic term (a) represents how fast HF-HRV raised or dropped; the linear term (b) represents the minimum HF-HRV can reach; and the constant (c) represents the initial level of HF-HRV. A quadratic instead of a linear model was chosen because an effective brain-regulated HF-HRV process is indexed by flexible and dynamic withdrawal and restoration of parasympathetic control with changing environmental demands.

(7) Physiological Data Collection & Processing

62. HRV is assessed using ECG (BIOPAC®). Standard electrodes can be placed using a standard lead-II electrode configuration. ECG is continuously monitored during the cognitive training process. HRV software (MINDWARE®) can be used to process data. A series of intervals between consecutive R waves (every 20 seconds) can be analyzed to generate HR, LF-HRV (0.04-0.15 Hz), HF-HRV (0.15-0.5 Hz), and LF/HF ratio. Averages of HRV data during the three phases can be computed separately. The primary measure of the proposed study is HF-HRV. In addition, a participant's pupil change and facial expression can be measured by facial video recording as another stream of physiological data that can provide complimentary information from what HRV can provide.

(8) Event-Related Potential (ERP):

63. ERP as an index measuring the neuroplasticity induced from the training activities, ERP data can also be collected through the electroencephalogram (EEG). Focus can be on N2pc and P3. EEG can be applied during the cognitive training process as well, along with ECG. The EEG data can be segmented into ERPs, and collected using 64 active scalp electrodes (Brain Products, LLC) at standard positions according to the 10-20 system. ERP amplitudes can be quantified using a signed area measure, which can tolerate individual and group differences in latency. ERP midpoint latencies can be quantified with a 50% area latency measure. The N2pc and P3 can be measured separately in their predefined time windows relative to target onset. N2pc and P3 have distinct sequential peaks in the waveform, which minimizes overlap of these two components and facilitates accurate measurement.

(9) Striatum-related networks analysis

64. The striatum-related network analysis was conducted in the following steps: first, bilateral striata were chosen as seeds according to Automated Anatomical Labeling template to calculate connectivity with voxels of the whole brain at baseline and post-training, respectively. Second, one sample t-test was applied to show the functional connectivity map of striatum in baseline with P<0.05 (False discovery rate, FDR-correction) and voxel size >50. In relation to the left striatum, three prefrontal regions were identified, including left inferior frontal gyms (−30, 42, 0), right middle frontal lobe (30, 30, 24), and left superior frontal gyrus (-3, 30, 54). In relation to the right striatum, three regions were found, including right inferior frontal gyrus (33, 39, −6), right superior frontal gyrus (9, 30, 54), and left superior frontal gyrus (−21, 33, 27). Training-induced changes in functional connectivity were examined using paired t-tests with a threshold of individual P<0.01, cluster size >1755 mm3, corresponding to corrected P<0.05. The correction was performed within the whole brain grey matter mask and determined with Monte Carlo simulations using the AFNI AlphaSim program. The analysis generated bilateral striatum-prefrontal networks.

(10) Resting-State Neuroimaging Data

65. Resting-state neuroimaging data was collected by acquiring two 5-minute BOLD functional scan with a gradient echo-planar imaging sequence (TR=2000 ms, TE=30 ms, 4 mm³ resolution, 30 axial slices). A 2D axial fast Gradient-Recalled Echo pulse sequence was used to generate field maps, which was used to correct for field inhomogeneity distortions in echo-planar imaging sequences. Two 5-min BOLD functional scans were acquired for each assessment period, using a gradient echo-planar imaging sequence (TR=2 s, TE=30 ms, 4 mm³ resolution, 30 axial slices). Participants were instructed to relax with their eyes open without falling asleep.

66. Resting-state neuroimaging data preprocessing consisted of motion correction, slice-timing correction, non-brain signal removal and Gaussian spatial smoothing (5 mm FWHM). Nuisance parameters (global, white matter and cerebrospinal fluid signals, motion) were removed through linear regression. Non-neuronal contributions were reduced with temporal filtering (0.01-0.08 Hz).

(11) Determine whether VSOP Training Improves Processing Speed and Attention and whether these Improvements are Associated with Changes of Brain Functional and Structural Connectivity.

67. Processing speed and attention are related to two neural networks: central executive network (CEN) and default mode network (DMN). These networks are significantly disrupted in MCI and are main markers for AD pathology. Compared to MLA, VSOP training leads to greater improvement in processing speed and attention (H1a), which is associated with better functional operation in these networks, indexed by more efficient resting state functional connectivity (H1b); and positive structural changes in these networks, indexed by improved white matter integrity using diffusion tensor imaging (DTI) (H1c).

(12) Test a novel neurophysiological pathway of VSOP training effects on brain structure and function.

68. Bidirectional links exist between the two neural networks described above and the autonomic nervous system (ANS). Importantly, the ANS, particularly the parasympathetic/vagal pathways, play a role in neuroplasticity in these regions. Further, vagal tone plays a key role in flexible adaptation to environmental stimuli, including tasks with heavy executive loads (e.g., cognitive training). According to the present findings, a sharp suppression, followed by an enhancement of vagal tone is linked with better cognitive and brain function; in turn, the training can modify resting state vagal activity, suggesting a reciprocal relationship. Compared to MLA, VSOP training induces greater ANS responses, indexed by a

U-shaped vagal control of ANS during training (H2a), and enhances the resting state vagal control of ANS after training (H2b), both of which relate to greater training-induced brain changes (H2c), and strengthen the association between changes of brain and processing speed and attention (H2d).

(13) Examine the Effect of VSOP Training on Untrained Cognitive and Functional Domains and the Role of Neurophysiological Changes Underlying these Possible Transfer Effects.

69. Transfer of learning from trained domains to untrained domains is the standard in evaluating the generalizability of improvement in cognitive training. Working memory, cognitive control, long-term memory, and instrumental activities of daily living are the cognitive and functional domains primarily affected in MCI and help differentiate MCI from AD. Training studies in AD-free older adults found inconsistent evidence for transfer effects of VSOP training. However, herein significant transfer effects of VSOP training in MCI were found. The two neural networks (CEN and DMN) provide both anatomical and functional platforms to support VSOP training transfer effects. Moreover, strengthening the striatum, a subcortical part of these brain networks, which is closely related to ANS responses, is critical for enhancing the transfer effects. Compared to MLA, VSOP training leads to improvements in multiple untrained cognitive and functional domains (H3a), which are associated with training-induced striatum changes (H3b); and U-shaped ANS responses strengthen the associations between cognitive performance in transfer domains and striatum changes (H3c).

b) Results

(1) Quadratic Model of HF-HRV Responses

70. In terms of model fit, the results for the VSOP group revealed a dynamic U-shaped HF-HRV response pattern, which was well fitted with a quadratic model. When applying the quadratic model to the VSOP group (FIG. 1), the parameter estimates were all significant (p<0.019). In contrast, for MLA group, only the constant was significant (p<0.001; FIG. 1A). In terms of group difference, the quadratic term, representing how “U-shaped” HF-HRV is, significantly differed between VSOP and MLA groups (t12=3.11, FDR-corrected P=0.027; no difference in the linear term, P=0.08, nor the constant, P=0.63, all tests two-tailed; FIG. 1B). Additionally, to further examine differences related to the U-shaped HF-HRV response, the raw

HF-HRV data between groups for two time points: in the middle (30th minute:) and the end (60th minute) was compared. A significant group difference was only observed at the end point, indicating a lack of the HF-HRV rebound for the MLA group (bootstrap resampling with replacement (n=1000) to modify the variance; 30th minute, mean difference =0.29, 95%Cl: −0.24, 0.78; 60th minute, mean difference =0.62, 95%Cl: 0.17, 1.05). Supporting the importance of the U-shaped HF-HRV response pattern, individual variation in the quadratic term was correlated with training-induced cognitive improvement, both in the trained domain (UFOV) and the transfer domain (working memory) using a bootstrap resampling with replacement (n=1000). Individual variation in the quadratic term correlated with changes in UFOV (r=0.39, 95%CI: 01, 0.70) and working memory (r=0.33, 95%CI: .06, 0.64). When only considering data from the

VSOP group, the correlation with UFOV remained significant (r=0.61, 95%CI: 0.10, 0.94), while the link with improvement in working memory was not significant (r=0.06, 95%CI: −0.72, −0.53). No significant correlations were found for the MLA control group (all |r|<0.04). Finally, changes in the striatum-prefrontal networks were correlated with the quadratic term of HF-HRV responses (Left: r=0.41, 95%CI: .19, 0 .91; Right: r=0.55, 95%CI: .39, 0.93).

71. Herein is shown that VSOP training yielded improvements in both trained (attention and processing speed, as measured by UFOV) and transferred (working memory) cognitive domains. Additional inquiries were made to determine if the observed improvements are related to HF-HRV. Supporting the importance of the U-shaped HF-HRV response pattern, individual variation in the quadratic term was correlated with training-induced cognitive improvement, both in the trained domain (UFOV) and the transfer domain (working memory). Across all participants, individual variation in the quadratic term correlated with changes in UFOV (r=0.39, 95%CI: 01, 0.70) and working memory (r=0.33, 95%CI: 0.06, .64). When only considering data from the VSOP group, the correlation with UFOV remained significant (r=0.61, 95%CI: 0.10, 0.94), while the link with improvement in working memory was not significant (r=0.06, 95%CI: −0.72, −0.53). No significant correlations were found for the MLA control group (all |r|<0.04). Taken together, the results reveal a consistent link between HF-HRV and training-induced improvements in UFOV—a task that has similar task demands as the VSOP intervention.

(2) Striatum-Related Network

72. Seed-based analysis generated two networks (Left striatum-Left inferior frontal gyrus (IFG) and Right striatum-Right middle frontal gyrus (MFG)) when taking bilateral striatum as the seeds (FIG. 2A). These striatum-prefrontal networks were differentially affected by VSOP and MLA training (FIGS. 2B and 2C; time (baseline and post-training) and group (VSOP and MLA) interaction: F_{1,20}=67.19, P<0.001, Partial η²=0.77). This was also the case when each striatum-prefrontal network was analyzed separately (both left and right stratum: P<0.001; FIG. 2C). Namely, decreased striatum-prefrontal connectivity was found as a result of VSOP training.

73. The changes in striatum-prefrontal connectivity were calculated from baseline to 7 week such that higher values indicated greater improvement after training. Changes in bilateral striatum-prefrontal networks were both significantly correlated to the changes in UFOV (Left: r=0.35, 5%CI: 0.05, 0.64; Right: r=0.55, 95%CI: .10, 0.88) and changes in working memory (Left: r=0.41, 95%CI: 0.22, 0.93; Right: r=0.55, 95%CI: 0.40, 0.92). Finally, changes in the striatum-prefrontal networks were correlated with the quadratic term of HF-HRV responses (Left: r=41, 95%CI: 0.19, .91; Right: r=0.55, 95%CI: 0.39, 0.93). Because of a smaller sample size for the neuroimaging analysis, we were not able to analyze data for each group separately.

c) Discussion

74. The present study provides neurophysiological evidence that both trained and untrained cognitive improvement of VSOP training reported previously can be explained by a flexible HF-HRV regulation in older adults with MCI. The results provide neurophysiological evidence that flexible PNS adaptation to cognitive training stimuli is associated with cognitive and neural improvements following VSOP training. This includes significant group differences in both HF-HRV and striatum-prefrontal connectivity, as well as significant correlational links between HF-HRV and both training-induced changes in UFOV and striatum-prefrontal connectivity. Specifically, compared to MLA group, VSOP training induced a more dynamic regulation of HF-HRV (indexed by the quadratic term of the HF-HRV response model), and such dynamic regulation was related to greater improvement in UFVO and working memory after training. A significant decrease in the strength of connectivity between bilateral striatum and frontal regions was also revealed, which corresponded to the dynamic regulation of HF-HRV pattern as well as the broad cognitive improvements after training. 75.

76. The nature of VSOP training, namely its attentional and processing demands, is well suited for stimulating PNS. This can explain both the group difference in HF-HRV and the correlation between HF-HRV and both the direct and transfer cognitive effects of training. For the correlations, those who are more attentive to the training would have both more dynamic HF-HRV responses and better training outcomes. This is further supported by a relatively strong correlation between HF-HRV and the direct training effect in VSOP group. This is significant as identifying easily measured, peripheral markers, like HF-HRV, that can index effective cognitive training is itself a worthwhile endeavor.

77. PNS carries information about viscerosensory states to the brain, predominantly striatum, in response to environmental stimuli. In response to environmental stimuli, a dynamic neurophysiological regulatory process occurs that promotes ongoing regulation and adaptation. This process is indexed by flexible withdrawal and restoration of PNS. VSOP training can be described as an intervention delivering a series of such novel environmental stimuli. Herein was shown that PNS response to VSOP training was characterized by a U-shaped HF-HRV response pattern that can be divided into two phases—phasic HRV suppression and enhancement. During the first suppression phase, a flexible withdrawal of HF-HRV occurred in response to VSOP training tasks. Such suppression is often seen in performing difficult mental stress tasks. The second enhancement phase occurred with adaptation to the task with a rebound involved. No such HF-HRV dynamics were found for the MLA control group. Importantly, such a U-shaped HF-HRV response was also associated with greater training effect on both trained (UFOV) and untrained (working memory) cognitive domains, which can be explained by the fact that monitoring PNS directly leads to improvements in cognitive processes by peripherally releasing neurohormones (e.g., noradrenalin, 5-hydroxytryptamine).

78. Empirical evidence indicates a link between PNS function and striatum, both at rest and in response to tasks. VSOP training in MCI resulted in reduced strength of connectivity between striatum and frontal regions. One explanation is that VSOP training helped enhance relevant neural efficiency in transferring information (e.g., releasing dopamine). Moreover, striatum provides an effective substrate for carrying transfer effects of VSOP training. The work provided herein also indicates that striatum is critical in supporting transfer effects of cognitive training, especially related to working memory.

### 2. Example 2

### Cognitive and Neural Effects of Vision-Based Speed-of-Processing Training in Older Adults with Amnestic Mild Cognitive Impairment

a) Methods

(1) Participants

79. This was a randomized, controlled, single-blinded trial. Participants with aMCI were recruited from the University of Rochester Memory Care Program (MCP) using the clinical diagnosis of MCI due to Alzheimer's disease. All participants had deficits in memory and executive function based on a comprehensive neuropsychological battery but intact activities of daily living and absence of dementia using the National Institute on Aging—Alzheimer's Association criteria according to assessments at MCP. Other inclusion criteria included stable use of AD medication, capacity to give consent based on clinician assessment, aged 60 and older, English speaking, adequate visual acuity for testing, and living in the community. Exclusion criteria included participation in another cognitive intervention study and active treatment with antidepressants or anxiolytics.

80. The University of Rochester Research subject review board approved the study. Twenty-four participants were enrolled and randomly assigned to the VSOP or MLA group after informed consent was provided and a baseline assessment was performed. Cognitive function and rsFC were assessed at baseline and after training. Interviewers were blinded to participants' group assignment. Three participants (2 from the VSOP group) withdrew after baseline assessment because of health concerns unrelated to the study. The baseline characteristics of the remaining 21 participants did not significantly differ between the two groups (Table 1).

TABLE 1
Baseline Sample Characteristics

| | Vision- | | |
|---|---|---|---|
| | Based | Mental | Independent |
| | Speed-of- | Leisure | T-Test or |
| | Processing | Activities | Chi-Square |
| | Training, | Control, | Test |
| Characteristic | n = 10 | n = 11 | (P-Value) |
| Age, mean ± SD | 72.9 ± 8.2 | 73.1 ± 9.6 | −0.05 (.96) |
| Male, n (%) | 5 (50.0) | 6 (54.5) | 0.04 (>.99) |
| Education high school or lower, | 1 (10.0) | 5 (45.5) | 3.23 (.15) |
| n (%) | | | |
| White, n (%) | 7 (70.0) | 10 (90.9) | 1.49 (.31) |
| 15-item Geriatric Depressive | 2.3 ± 1.9 | 3.6 ± 0.7 | −1.39 (.18) |
| Scale score, mean ± SD | | | |
| History of engaging in mental | 3.8 ± 0.7 | 4.44 ± 1.0 | −1.56 (.14) |
| leisure activities, mean ± SD\hat{ } | | | |
| Montreal Cognitive Assessment | 24.4 ± 2.6 | 25.6 ± 1.6 | −1.25 (.23) |
| score, mean ± SD | | | |

SD = standard deviation.
\hat{ }the questionnaire asked the frequency of engaging in 6 types of activities (e.g., reading, playing cards, attending lectures) using a 6-point ordinal scale ranging from 1 (daily) to 6 (never); an average score was calculated.

(2) Intervention

81. VSOP training used the INSIGHT online program (Posit Science, San Francisco, Calif.), which included five training tasks: eye for detail, peripheral challenge, visual sweeps, double decision, and target tracker. Participants responded by identifying what object they saw or where they saw it on the screen. The training automatically adjusted the task difficulty and speed based on the participant's performance, ensuring that participants always operated near their optimal capacity. The completion percentage and score of each task were recorded. Training performance was calculated relative to the normative data from the Posit Science database and expressed as a percentile. As expected, VSOP training resulted in significant performance increases (pre-training mean: 34.4±13.2%; post-training mean: 52.2±16.5%; Wilcoxon test: Z=−2.81, P=0.005).

82. MLA control activities were chosen to control for computer and online experience and amount of time, simulate participants' everyday mental activities, and entertain participants to prevent dropping out. Online crossword, Sudoku, and solitaire games were used. Participants could choose to practice any combination of these games. Both groups were asked to practice 1 hour per day 4 days per week for 6 weeks in their homes. Hours spent on training tasks were recorded in both groups; no significant differences were found (VSOP: 15.4±6.6 hours; MLA: 19.3±8.1, t20=−1.14, P=0.27). There were no correlations between training duration and training effects reported below in the entire sample (all P>0.10). Of note, in VSOP training studies of healthy older adults, typical training duration is approximately 10 hours.

(3) Cognitive Measures

83. The Useful Field of View (UFOV) is a computerized test assessing visual processing speed and attention. Visual and attentional demands of UFOV are similar (although not identical) to the task demands in VSOP training. A composite score of UFOV was developed by averaging the reaction times of three individual tasks (processing speed, selective attention, divided attention). The use of the composite score is consistent with the approach used in other clinical trials.

84. The EXAMINER is a computerized test designed for clinical trials that measures three executive function domains: cognitive control (set shifting and flanker tasks), verbal fluency (phonemic and categorical fluency), and working memory (dot counting and 1-back). This three domain model was determined using confirmatory factor analysis, and the generation of composite scores was based on item response theory methods. EXAMINER uses several comparable assessment packages to avoid using identical tests at different assessment points.

85. Timed instrumental activities of daily living (TIADL) objectively measure performance speed and accuracy on multiple IADL domains. It is more sensitive measurement than the traditional self-report instruments in detecting subtle decline in everyday function in persons with MCI. Time spent on each task was recorded, with adjustment on whether an individual accurately completed each task. Average completion time of the tasks was used as the outcome measure.

86. Neuroimaging data were acquired using magnetic resonance imaging (TimTrio 3T system, Siemens, Erlangen, Germany) using a 32-channel head coil. High-resolution T1-weighted structural images were acquired using MPRAGE (inversion time =950 ms, echo time (TE) =3.87 ms, repetition time (TR)=1,620 ms, 1-mm³ resolution). A two-dimensional axial fast gradient-recalled echo pulse sequence was used to generate field maps, which were used to correct for field inhomogeneity distortions in echo-planar imaging sequences. Two 5-minute blood-oxygen-level-dependent functional scans were acquired for each assessment period using a gradient echoplanar imaging sequence (TR=2 seconds, TE=30 ms, 4-mm³ resolution, 30 axial slices). Participants were instructed to relax with their eyes open without falling asleep.

87. rsFC data were analyzed using the FSL software. Data preprocessing consisted of motion correction, slice-timing correction, non-brain signal removal, and Gaussian spatial smoothing (5-mm full width at half maximum). Nuisance parameters (global, white matter and cerebrospinal fluid signals, motion) were removed using linear regression. Nonneuronal contributions were reduced using temporal filtering (0.01-0.08 Hz). The Multivariate Exploratory Linear Optimized Decomposition into Independent Components algorithm was used to generate resting state networks. The DMN and CEN were identified based on previous literature. Network-specific regions of interest (ROIs) were selected using the Harvard-Oxford Atlas. Correlation of time courses between all possible pairs of within-network ROIs were computed and Fisher Z-transformed, with the average correlation coefficient representing the strength of the network.

88. Other data analysis was conducted using SPSS 21.0 (SPSS, Inc., Chicago, Ill.). To examine group differences at baseline, independent t-tests were conducted for continuous variables and chi-square tests for categorical variables. The Wilcoxon test was used to examine within-group effects of training. Baseline cognitive and neural outcomes did not significantly differ between the two groups except that participants in the VSOP training had worse working memory (P=0.03). A repeated-measures general linear model was used to examine between-groups effects of training; the main and interacted terms of time and group were examined when controlling for baseline differences. For reported P-values, false-discovery rate was used to address for multiple comparisons across outcomes.

89. The sample size was based on a previous VSOP training study of multiple-domain aMCI, which reported an effect size (η2) of 0.37 when comparing post-training UFOV with a no-contact control group. From this result, it was estimated that the minimum total sample size would be 14 (based on a =0.05, power =0.80, two groups, two repeated measures, and 0.50 correlation between repeated measures). This compares favorably with the total sample size of 21.

b) Results

(1) Training Effects on Trained and Transferred Cognitive Outcomes

90. Within-group cognitive changes were first examined (FIGS. 3A, B, Table 2), contrasting baseline with post-training outcomes. For the VSOP group, significant improvements were found in the trained domain (UFOV, Z=−2.70, P=0.007) and two transfer domains (working memory: Z=2.31, P=0.02, and IADL: Z=−2.29, P=0.02) but no significant changes in two other transfer domains (verbal fluency and cognitive control). For the MLA group, there were no significant improvements (all P≧0.10).

TABLE 2
Baseline and Posttraining Cognitive and Neural Scores by Group

| | | | | | | | Group × Time | |
|---|---|---|---|---|---|---|---|---|
| | Vision-Based Speed-of-Processing | | | | | | interaction^{d} | |
| Cognitive and Neural | Training (n = 10) | | | Mental Leisure Activities Control (n = 11) | | | | Partial |
| Outcomes | Baseline | Posttraining | Z (P-Value)^{c} | Baseline | Posttraining | Z (P-Value)^{e} | F (P-Value) | η² |
| Useful field of view^{a} | 136.3 ± 87.4 | 64.0 ± 22.2 | −2.70 (.007)^{e} | 96.6 ± 48.7 | 87.6 ± 59.5 | −1.33 (.18) | 6.61 (.02)^{e} | 0.26 |
| Working memory^{b} | −0.58 ± 0.71 | 0.11 ± 0.37 | 2.31 (.02)^{e} | 0.26 ± 0.68 | −0.06 ± 0.76 | −.98 (.33) | 7.33 (.01)^{e} | 0.28 |
| Verbal fluency^{b} | 0.55 ± 0.48 | 0.50 ± 0.57 | −0.46 (.65) | 0.34 ± 0.69 | 0.21 ± 0.70 | −0.18 (.86) | 0.09 (.77) | 0.005 |
| Cognitive control^{b} | 0.21 ± 0.46 | 0.26 ± 0.38 | 0.68 (.50) | 0.38 ± 0.58 | 0.49 ± 0.68 | 1.26 (.21) | 0.14 (.71) | 0.008 |
| Instrumental activities | 19.8 ± 6.6 | 14.6 ± 4.2 | −2.29 (.02)^{e} | 14.2 ± 4.6 | 15.4 ± 4.5 | 0.71 (.48) | 5.16 (.03)^{e} | 0.21 |
| of daily living^{a} | | | | | | | | |
| Central executive | 0.77 ± 0.23 | 0.47 ± 0.17 | −2.37 (.02)^{e} | 0.62 ± 0.26 | 0.45 ± 0.17 | −1.46 (.14) | 2.03 (.19) | 0.04 |
| network^{a} | | | | | | | | |
| Default mode | 0.70 ± 0.14 | 0.73 ± 0.16 | 1.04 (.31) | 0.63 ± 0.18 | 0.45 ± 0.18 | −1.83 (.07) | 14.63 (.004)^{e} | 0.62 |
| network^{b} | | | | | | | | |

^{a}Higher is worse.
^{b}Lower is worse.
^{c}Within-group comparison using Wilcoxon test.
^{d}Between-group comparison using repeated-measures general linear model controlled for group and main effects of time.
^{e}Significant level remained after false discovery rate adjustment.

91. The same pattern of results was evident in between group comparisons (FIGS. 3A, 3B, Table 2). The VSOP group exhibited significantly greater improvements in UFOV (group-by-time interaction F1, 19=6.61, partial g2=0.26, P=0.02), working memory (group-by-time interaction F1, 19 =7.33, partial g2 =0.28, P=0.01), and IADL (group-by-time interaction F1, 19=5.16, partial g2=0.21, P=0.03) than the MLA group.

(2) Training Effects on Resting-State Neural Networks

92. For the VSOP group, significant improvement was found in CEN connectivity (Z=2.37, P=0.02, as indexed according to poor connectivity strength) and no change in DMN (FIG. 3C, Table 2). The MLA group showed no CEN changes and a trend for worsening of DMN (Z=1.83, P=0.07, as indexed according to poor strength of connectivity). Between-group analysis (FIG. 3C, Table 2) showed that VSOP training resulted in significantly greater improvements than MLA (indexed according to greater connectivity) in the DMN (group-by-time interaction F1, 9=14.63, partial g2=0.62, P=0.004) but not CEN. A summary of the results is presented in Table 2.

c) Discussion

93. The present study shows that, in addition to the improvement in the trained domain, VSOP training led to improvements in working memory and IADLs. The results also link VSOP training with maintenance of DMN connectivity strength and a decrease in CEN connectivity.

94. The transfer of VSOP training to untrained cognitive and functional domains is of clinical significance. There are several nonexclusive explanations of this transfer effect. First, because individuals with MCI have low baseline cognitive capacity, they have more room for improvement in the trained and untrained domains. Second, the VSOP training used here includes a rich combination of visual processing speed and attention tasks (see Methods). This is in contrast to previous studies that relied on a single task, although transfer effects of the training exhibited a certain degree of specificity. For example, significant changes were not found in verbal fluency, which is probably due to the lack of linguistic stimuli in the training tasks. The specificity of transfer effects across different executive function domains requires further investigation with larger sample sizes.

95. The two brain networks examined in the present study provide a possible functional platform for disseminating training effects from one region to another. VSOP training in MCI was linked with lower CEN connectivity and maintenance of DMN connectivity. One explanation for the lower CEN connectivity is that VSOP training helped enhance the efficiency of information processing, which reduced the frontal lobe-oriented dependence. Weakening of DMN connectivity is a consistently identified marker of neurodegeneration. Although the VSOP training did not enhance DMN connectivity, maintenance of DMN connectivity can be viewed as a positive outcome given naturally worsening processes in MCI. Supporting this argument, a trend for weakened DMN connectivity in the MLA group was found. This is not surprising, because a recent cohort study found MLA to be independent of brain pathology.

### B. REFERENCES

Ahs, F., Sollers, J. J., 3rd, Furmark, T., Fredrikson, M., and Thayer, J. F. (2009). High-frequency heart rate variability and cortico-striatal activity in men and women with social phobia. NeuroImage 47, 815-820.

Ahs, F., Sollers, J. J., 3rd, Furmark, T., Fredrikson, M., and Thayer, J. F. (2009). High-frequency heart rate variability and cortico-striatal activity in men and women with social phobia. NeuroImage 47, 815-820.

Albert, M. S., DeKosky, S. T., Dickson, D., Dubois, B., Feldman, H. H., Fox, N. C., Gamst, A., Holtzman, D. M., Jagust, W. J., Petersen, R. C., et al. (2011). The diagnosis of mild cognitive impairment due to Alzheimer's disease: recommendations from the National Institute on Aging-Alzheimer's Association workgroups on diagnostic guidelines for Alzheimer's disease. Alzheimers Dement 7, 270-279.

Albert, M. S., DeKosky, S. T., Dickson, D., Dubois, B., Feldman, H. H., Fox, N. C., Gamst, A., Holtzman, D. M., Jagust, W. J., Petersen, R. C., et al. (2011). The diagnosis of mild cognitive impairment due to Alzheimer's disease: recommendations from the National Institute on Aging-Alzheimer's Association workgroups on diagnostic guidelines for Alzheimer's disease. Alzheimers Dement 7, 270-279.

Anguera J A, Boccanfuso J, Rintoul J L, Al-Hashimi O, Faraji F, Janowich J, et al. Video game training enhances cognitive control in older adults. Nature 501(7465): 97-101.(2013).

Ball K, Edwards J D, Ross L A. The impact of speed of processing training on cognitive and everyday functions. J Gerontol B Psychol Sci Soc Sci. 2007;62 Spec No 1:19-31.

Ball K, Edwards J D, Ross L A. The impact of speed of processing training on cognitive and everyday functions. J Gerontol B Psychol Sci Soc Sci. 2007;62 Spec No 1:19-31.

Bavelier D, Green C S, Pouget A, Schrater P. Brain plasticity through the life span: learning to learn and action video games. Annual review of neuroscience 35: 391-416.(2012).

Beacher, F. D., Gray, M. A., Mathias, C. J., and Critchley, H. D. (2009). Vulnerability to simple faints is predicted by regional differences in brain anatomy. NeuroImage 47, 937-945.

Beacher, F. D., Gray, M. A., Mathias, C. J., and Critchley, H. D. (2009). Vulnerability to simple faints is predicted by regional differences in brain anatomy. NeurozImage 47, 937-945.

Beissner, F., Meissner, K., Bar, K. J., and Napadow, V. (2013). The autonomic brain: an activation likelihood estimation meta-analysis for central processing of autonomic function. J Neurosci 33, 10503-10511.

Beissner, F., Meissner, K., Bar, K. J., and Napadow, V. (2013). The autonomic brain: an activation likelihood estimation meta-analysis for central processing of autonomic function. J Neurosci 33, 10503-10511.

Belleville S, Clement F, Mellah S, Gilbert B, Fontaine F, Gauthier S. Training-related brain plasticity in subjects at risk of developing Alzheimer's disease. Brain 134(Pt 6): 1623-34.(2011).

Clark, K. B., Naritoku, D. K., Smith, D. C., Browning, R. A., and Jensen, R. A. (1999). Enhanced recognition memory following vagus nerve stimulation in human subjects. Nat Neurosci 2, 94-98.

Clark, K. B., Naritoku, D. K., Smith, D. C., Browning, R. A., and Jensen, R. A. (1999). Enhanced recognition memory following vagus nerve stimulation in human subjects. Nat Neurosci 2, 94-98.

Dahlin, E., Neely, A. S., Larsson, A., Backman, L., and Nyberg, L. (2008). Transfer of learning after updating training mediated by the striatum. Science 320, 1510-1512.

Dahlin, E., Neely, A. S., Larsson, A., Backman, L., and Nyberg, L. (2008). Transfer of learning after updating training mediated by the striatum. Science 320, 1510-1512.

Daviglus, M. L., Bell, C. C., Berrettini, W., Bowen, P. E., Connolly, E. S., Jr., Cox, N. J., Dunbar-Jacob, J. M., Granieri, E. C., Hunt, G., McGarry, K., et al. (2010). National Institutes of Health State-of-the-Science Conference statement: preventing alzheimer disease and cognitive decline. Annals of Internal Medicine 153, 176-181.

Daviglus, M. L., Bell, C. C., Berrettini, W., Bowen, P. E., Connolly, E. S., Jr., Cox, N. J., Dunbar-Jacob, J. M., Granieri, E. C., Hunt, G., McGarry, K., et al. (2010). National Institutes of Health State-of-the-Science Conference statement: preventing alzheimer disease and cognitive decline. Annals of Internal Medicine 153, 176-181.

Erickson, K. I., Boot, W. R., Basak, C., Neider, M. B., Prakash, R. S., Voss, M. W., Graybiel, A M., Simons, D. J., Fabiani, M., Gratton, G., et al. (2010). Striatal volume predicts level of video game skill acquisition. Cereb Cortex 20, 2522-2530.

Erickson, K. I., Boot, W. R., Basak, C., Neider, M. B., Prakash, R. S., Voss, M. W., Graybiel, A M., Simons, D. J., Fabiani, M., Gratton, G., et al. (2010). Striatal volume predicts level of video game skill acquisition. Cereb Cortex 20, 2522-2530.

Groves, D. A., and Brown, V. J. (2005). Vagal nerve stimulation: a review of its applications and potential mechanisms that mediate its clinical effects. Neurosci Biobehav Rev 29, 493-500.

Groves, D. A., and Brown, V. J. (2005). Vagal nerve stimulation: a review of its applications and potential mechanisms that mediate its clinical effects. Neurosci Biobehav Rev 29, 493-500.

Kiesel A, Miller J, Jolicoeur P, Brisson B. Measurement of ERP latency differences: a comparison of single-participant and jackknife-based scoring methods. Psychophysiology. 2008;45(2):250-274.

Kiesel A, Miller J, Jolicoeur P, Brisson B. Measurement of ERP latency differences: a comparison of single-participant and jackknife-based scoring methods. Psychophysiology. 2008;45(2):250-274.

Lane, R. D., McRae, K., Reiman, E. M., Chen, K., Ahem, G. L., and Thayer, J. F. (2009). Neural correlates of heart rate variability during emotion. NeuroImage 44, 213-222.

Lane, R. D., McRae, K., Reiman, E. M., Chen, K., Ahern, G. L., and Thayer, J. F. (2009). Neural correlates of heart rate variability during emotion. NeuroImage 44, 213-222.

Ledberg, A., Akerman, S., and Roland, P. E. (1998). Estimation of the probabilities of 3D clusters in functional brain images. Neuroimage 8, 113-128.

Ledberg, A., Akerman, S., and Roland, P. E. (1998). Estimation of the probabilities of 3D clusters in functional brain images. Neuroimage 8, 113-128.

Lee K A, Hicks G. Nino-Murcia G. Validity and reliability of a scale to assess fatigue. Psychiatry Res. 1991;36(3):291-298.

Lee K A, Hicks G, Nino-Murcia G. Validity and reliability of a scale to assess fatigue. Psychiatry Res. 1991;36(3):291-298.

Lin F, Chen D, Vance D E, Mapstone M. Trajectories of Combined Laboratory- and Real World-based Speed of Processing in Community-Dwelling Older adults: Predictors and Functional Outcomes Journal of Gerontology: Psychological Sciences and Social Sciences. 2012.

Lin F, Chen D, Vance D E, Mapstone M. Trajectories of Combined Laboratory- and Real World-based Speed of Processing in Community-Dwelling Older adults: Predictors and Functional Outcomes Journal of Gerontology: Psychological Sciences and Social Sciences. 2012.

Lin F, Cole D, Cotton K, et al. Executive Homeostatic Network in Mild Cognitive Impairment (MCI) Translational Science 2014 Meeting; April 9-11, 2014; Washington, DC.

Lin F, Cole D, Cotton K, et al. Executive Homeostatic Network in Mild Cognitive Impairment (MCI) Translational Science 2014 Meeting; April 9-11, 2014; Washington, DC.

Lovden M, Backman L, Lindenberger U, Schaefer S, Schmiedek F. A theoretical framework for the study of adult cognitive plasticity. Psychol Bull. 2011;136(4):659-676.

Lovden M, Backman L, Lindenberger U, Schaefer S, Schmiedek F. A theoretical framework for the study of adult cognitive plasticity. Psychol Bull. 2011;136(4):659-676.

Nako R, Wu R, Eimer M. Rapid guidance of visual search by object categories. Journal of experimental psychology. Human perception and performance. 2014;40(1):50-60.

Nako R, Wu R, Eimer M. Rapid guidance of visual search by object categories. Journal of experimental psychology. Human perception and performance. 2014;40(1):50-60.

O'Brien J L, Edwards J D, Maxfield N D, Peronto C L, Williams V A, Lister J J. Cognitive training and selective attention in the aging brain: an electrophysiological study. Clin Neurophysiol 124(11): 2198-208.(2013).

Ogbonnaya, S., and Kaliaperumal, C. (2013). Vagal nerve stimulator: Evolving trends. J Nat Sci Biol Med 4, 8-13.

Ogbonnaya, S., and Kaliaperumal, C. (2013). Vagal nerve stimulator: Evolving trends. J Nat Sci Biol Med 4, 8-13.

Ohira, H., Matsunaga, M., Osumi, T., Fukuyama, S., Shinoda, J., Yamada, J., and Gidron, Y. (2013). Vagal nerve activity as a moderator of brain-immune relationships. J Neuroimmunol 260, 28-36.

Ohira, H., Matsunaga, M., Osumi, T., Fukuyama, S., Shinoda, J., Yamada, J., and Gidron, Y. (2013). Vagal nerve activity as a moderator of brain-immune relationships. J Neuroimmunol 260, 28-36.

Park, G., Vasey, M. W., Van Bavel, J. J., and Thayer, J. F. (2014). When tonic cardiac vagal tone predicts changes in phasic vagal tone: the role of fear and perceptual load. Psychophysiology 51, 419-426.

Park, G., Vasey, M. W., Van Bavel, J. J., and Thayer, J. F. (2014). When tonic cardiac vagal tone predicts changes in phasic vagal tone: the role of fear and perceptual load. Psychophysiology 51, 419-426.

Porges, S. W. (1995). Orienting in a defensive world: Mammalian modifications of our evolutionary heritage: A Polyvagal Theory. Psychophysiology 32, 301-318.

Porges, S. W. (1995). Orienting in a defensive world: Mammalian modifications of our evolutionary heritage: A Polyvagal Theory. Psychophysiology 32, 301-318.

Porges, S. W. (2003). The Polyvagal Theory: Phylogenetic contributions to social behavior. Physiol. Behav. 79, 503-513.

Porges, S. W. (2003). The Polyvagal Theory: Phylogenetic contributions to social behavior. Physiol. Behav. 79, 503-513.

Possin KL, Feigenbaum D, Rankin K P, Smith G E, Boxer A L, Wood K, et al. Dissociable executive functions in behavioral variant frontotemporal and Alzheimer dementias. Neurology2013).

Rebok G W, Ball K, Guey L T, Jones R N, Kim H Y, King J W, et al. Ten-Year Effects of the Advanced Cognitive Training for Independent and Vital Elderly Cognitive Training Trial on Cognition and Everyday Functioning in Older Adults. J Am Geriatr Soc2014).

Ren P, Mapstone M, Wu R, Lin F. The Resting State Neural Basis for Intra-Individual Variability in Reaction Time in Mild Cognitive Impairment. under review.

Ren P, Mapstone M, Wu R, Lin F. The Resting State Neural Basis for Intra-Individual Variability in Reaction Time in Mild Cognitive Impairment. under review.

Ren P, Wu R, Mapstone M, Lin F. Altered Functional Connectivity of Prefrontal Cortex Underpins the Abnormality of Intra-Individual Variability in Reaction Time in Mild Cognitive Impairment. under review.

Ren P, Wu R, Mapstone M, Lin F. Altered Functional Connectivity of Prefrontal Cortex Underpins the Abnormality of Intra-Individual Variability in Reaction Time in Mild Cognitive Impairment. under review.

Saint-Cyr, J. A. (2003). Frontal-striatal circuit functions: context, sequence, and consequence. J Int Neuropsychol Soc 9, 103-127.

Saint-Cyr, J. A. (2003). Frontal-striatal circuit functions: context, sequence, and consequence. J Int Neuropsychol Soc 9, 103-127.

Sanz-Arigita E J, Schoonheim M M, Damoiseaux J S, Rombouts S A, Maris E, Barkhof F, et al. Loss of ‘small-world’ networks in Alzheimer's disease: graph analysis of FMRI resting-state functional connectivity. PLoS One 5(11): e13788.(2010).

Sawaki R, Geng J J, Luck S J. A common neural mechanism for preventing and terminating the allocation of attention. J Neurosci. 2012;32(31):10725-10736.

Sawaki R, Geng J J, Luck S J. A common neural mechanism for preventing and terminating the allocation of attention. J Neurosci. 2012;32(31):10725-10736.

Schlindwein, P., Buchholz, H. G., Schreckenberger, M., Bartenstein, P., Dieterich, M., and Birklein, F. (2008). Sympathetic activity at rest and motor brain areas: FDG-PET study. Auton Neurosci 143, 27-32.

Schlindwein, P., Buchholz, H. G., Schreckenberger, M., Bartenstein, P., Dieterich, M., and Birklein, F. (2008). Sympathetic activity at rest and motor brain areas: FDG-PET study. Auton Neurosci 143, 27-32.

Supekar K, Menon V, Rubin D, Musen M, Greicius M D. Network analysis of intrinsic functional brain connectivity in Alzheimer's disease. PLoS computational biology 4(6): e1000100.(2008).

Surowka A D, Krygowska-Wajs A, Ziomber A, Thor P, Chrobak A A, Szczerbowska-Boruchowska M. Peripheral vagus nerve stimulation significantly affects lipid composition and protein secondary structure within dopamine-related brain regions in rats. Neuromolecular Med 17(2): 178-91.(2015).

Thayer, J. F., Ahs, F., Fredrikson, M., Sollers, J. J., 3rd, and Wager, T. D. (2012). A meta-analysis of heart rate variability and neuroimaging studies: implications for heart rate variability as a marker of stress and health. Neurosci Biobehav Rev 36, 747-756.

Thayer, J. F., Ahs, F., Fredrikson, M., Sollers, J. J., 3rd, and Wager, T. D. (2012). A meta-analysis of heart rate variability and neuroimaging studies: implications for heart rate variability as a marker of stress and health. Neurosci Biobehav Rev 36, 747-756.

Thayer, J. F., and Lane, R. D. (2000). A model of neurovisceral integration in emotion regulation and dysregulation. J Affect Disord 61, 201-216.

Thayer, J. F., and Lane, R. D. (2000). A model of neurovisceral integration in emotion regulation and dysregulation. J Affect Disord 61, 201-216.

Thayer, J. F., Hansen, A. L., Saus-Rose, E., and Johnsen, B. H. (2009). Heart rate variability, prefrontal neural function, and cognitive performance: the neurovisceral integration perspective on self-regulation, adaptation, and health. Ann Behav Med 37, 141-153.

Thayer, J. F., Hansen, A. L., Saus-Rose, E., and Johnsen, B. H. (2009). Heart rate variability, prefrontal neural function, and cognitive performance: the neurovisceral integration perspective on self-regulation, adaptation, and health. Ann Behav Med 37, 141-153.

Weber, C. S., Thayer, J. F., Rudat, M., Wirtz, P. H., Zimmermann-Viehoff, F., Thomas, A., Perschel, F. H., Arck, P. C., and Deter, H. C. (2010). Low vagal tone is associated with impaired post stress recovery of cardiovascular, endocrine, and immune markers. European journal of applied physiology 109, 201-211.

Weber, C. S., Thayer, J. F., Rudat, M., Wirtz, P. H., Zimmermann-Viehoff, F., Thomas, A., Perschel, F. H., Arck, P. C., and Deter, H. C. (2010). Low vagal tone is associated with impaired post stress recovery of cardiovascular, endocrine, and immune markers. European journal of applied physiology 109, 201-211.

Wu R, Scerif G, Aslin RN, Smith T J, Nako R, Eimer M. Searching for something familiar or novel: top-down attentional selection of specific items or object categories. J Cogn Neurosci. 2013;25(5):719-729.

Wu R, Scerif G, Aslin R N, Smith T J, Nako R, Eimer M. Searching for something familiar or novel: top-down attentional selection of specific items or object categories. J Cogn Neurosci. 2013;25(5):719-729.

Yeh, T. L., Yang, Y. K., Chiu, N. T., Yao, W. J., Yeh, S. J., Wu, J. S., Chuang, J. I., and Chang, S. H. (2006). Correlation between striatal dopamine D2/D3 receptor binding and cardiovascular activity in healthy subjects. Am J Hypertens 19, 964-969.

Yeh, T. L., Yang, Y. K., Chiu, N. T., Yao, W. J., Yeh, S. J., Wu, J. S., Chuang, J. I., and Chang, S. H. (2006). Correlation between striatal dopamine D2/D3 receptor binding and cardiovascular activity in healthy subjects. Am J Hypertens 19, 964-969.

What is claimed is:

1. A cognitive training system for administering cognitive training comprising a computer, a training module specifically designed to administer a cognitive training program and receive training data, a display for administration of the training program, an input device for receiving patient training data, a portable high frequency heart rate variability (HF-HRV) monitor, a receiver configured to receive input from the variable heart rate monitor, a communication module specifically designed to convert the signal from the monitor into useable input data for use in the training program; wherein the cognitive training system continually adjusts the training based on input from the HF-HRV monitor to maximize cognitive plasticity.

2. The cognitive training system of claim 1, wherein the HF-HRV monitor comprises one or more electrode leads for measuring cardiac electrical potential.

3. The cognitive training system of claim 2, wherein the receiver has inputs to receive signals from the HF-HRV monitor.

4. The cognitive training system of claim 2, wherein the HF-HRV monitor further comprises a transmitter module which receives HF-HRV data and transmits them to the receiver.

5. The cognitive training system of claim 4, wherein in the transmitter module is integrated into the monitor.

6. The cognitive training system of claim 4, wherein in the transmitter module is physically separate from the monitor.

7. The cognitive training system of claim 1, wherein the HF-HRV monitor comprises a light source and an optical sensor to measure light absorbance or reflectivity of the light off of capillaries in the subject.

8. The cognitive training system of claim 7, wherein the receiver has inputs to receive signals from the HF-HRV monitor.

9. The cognitive training system of claim 7, wherein the HF-HRV monitor further comprises a transmitter module which receives HF-HRV data and transmits them to the receiver.

10. The cognitive training system of claim 9, wherein in the transmitter module is integrated into the monitor.

11. The cognitive training system of claim 9, wherein in the transmitter module is physically separate from the monitor.

12. The cognitive training system of claim 1, wherein the monitor communicates with the receiver wirelessly.

13. The cognitive training system of claim 1, wherein the monitor is wearable.

14. The cognitive training system of claim 1, wherein the communication module is a component of the training module.

15. The cognitive training system of claim 1, wherein the communication module is a component of the receiver.

16. A method of treating a subject with mild cognitive impairment comprising administering cognitive training to the subject, measuring high frequency variable heart rate of the subject, correlating the HF-HRV measurement with the neural plasticity of the subject, modulating the difficulty of the cognitive training to optimize plasticity of the subject induced by the cognitive training.

17. The method of claim 16, wherein the cognitive training program comprises vision-based speed of processing cognitive training.

18. The method of claim 16, wherein the HF-HRV measurement and adjustments to the difficulty of the cognitive training are performed continuously throughout the training.

19. The method of claim 16, wherein the adjustments to the cognitive training are performed automatically by a cognitive training system.

20. The method of claim 16, wherein the adjustments to the cognitive training are made by a practitioner administering the cognitive training program.
